(19) United States
(12) Patent Application Publication
(10) Pub. No.: US 2021/0093693 A1
(43) Pub. Date: Apr. 1, 2021

(54) NOVEL USE OF GIT HAVING ANTI-SENESCENCE ACTIVITY

(71) Applicant: Chungbuk National University Industry-Academic Cooperation Foundation, Chungcheongbuk-do (KR)

(72) Inventors: Eung Gook Kim, Chungcheongbuk-do (KR); Eun Young Shin, Chungcheongbuk-do (KR)

(21) Appl. No.: 16/947,427

(22) Filed: Jul. 31, 2020

(30) Foreign Application Priority Data
Oct. 1, 2019 (KR) 10-2019-0121825

(51) Int. Cl. A61K 38/17

(52) U.S. Cl. CPC A61K 38/177; A61K 48/00

(57) ABSTRACT

Disclosed herein are a G-protein-coupled receptor (GPCR) kinase-interacting protein (GIT) and a use thereof. More particularly, a pharmaceutical composition for prevention or treatment of senescence or senescence-associated disease and a composition for inhibition of cellular senescence, each comprising GIT or a GIT activating agent as an active ingredient, a method for screening a cellular senescence inhibitor, and a method for inhibition of cellular senescence are provided. In senescent cells induced by down-regulation of bPIX expression, GIT or GIT-CT overexpression was found to reduce the expression of senescence markers and to suppress senescence-induced endocytosis reduction. Having an inhibitory activity against cellular senescence, GIT can be thus used as a novel therapeutic agent for senescence or senescence-associated disease.

### CROSS-REFERENCE TO RELATED APPLICATIONS

This application claims priority under the Paris Convention to foreign Korean Application No. 10-2019-0121825, filed Oct. 1, 2019, which is herein incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Oct. 6, 2020, is named 2020-10-06_KIM_P13294US00_SEQLISTING_ST25.txt and is 31,743 bytes in size.

### BACKGROUND OF THE INVENTION

### 1. Field of the Invention

The present disclosure relates to a novel use of GIT protein having anti-senescence activity.

### 2. Description of the Prior Art

Cellular senescence is a main cause for organismal aging and is induced by many factors including accumulated DNA damage, telomere erosion, cellular stress, loss of cell reproduction ability, and so on. Senescent cells are characterized by morphological traits such as enlarged sizes, flattened shapes, elevated nuclear heterochromatin levels, abundant cytosolic vacuoles, etc. In addition, representative biochemical hallmarks of senescence include elevated senescence-associated β-galactosidase (SA-β-Gal) activity, increased expression levels of cell growth inhibiting molecules such as p53, p16/INK4 (p16), and p21, and increased secretion of inflammation-related proteins such as insulin-like growth factor binding proteins (IGFBPs), interleukin-6, transforming growth factor-θ (TGF-β), and interferon.

In addition to simply contributing to organismal or tissue aging, cellular senescence plays an important role in the pathology of various diseases. Senescent cells are abundantly found in inflammatory lesion tissues such as tissues affected by rheumatoid arthritis, osteoarthritis, hepatitis, chronic dermal injury, vascular tissues affected by arteriosclerosis vascular, etc. Cellular senescence is also observed upon benign prostatic hyperplasia, hepatitis, liver cancer, etc.

When senescent cells are accumulated, the cells are less prone to undergoing cell division, resulting in inability to appropriately repair the injured tissues. Furthermore, senescent cells promotes the secretion of histolytic enzymes or inflammatory cytokines to accelerate the injury of surrounding tissues, contributing to the onset of senescence-associated disease. Hence, because cellular senescence closely correlates with organismal aging, active research on controlling organismal aging by delaying cellular senescence has been ongoing.

Senescent cells have been generally characterized to remarkably decrease in responsiveness to external stimuli and have a lowered receptor-mediated endocytosis function. In this regard, there was a report on a transferrin uptake assay through which an observation was made of noticeably poor receptor-mediated endocytosis in senescent cells, compared to presenescent cells. Receptor-mediated endocytosis is known to progress via clathrin-coated vesicles in multiple steps in which clathrin, AP2, dynamin, and amphiphysin are involved.

In addition, senescent cells have a hyper-adhesive cell phenotype, showing strong adhesion to substrates through large focal adhesions (FAs), activated FA kinase (FAK), etc.

G-protein-coupled receptor (GPCR) kinase-interacting protein (GIT) is a multifunctional adaptor protein composed of several domains including ARF GTPase-activating protein domain (ARF GAP domain) and is known to associate with various signaling proteins and adaptor proteins such as GRK, PIX, FAK, PLCγ, MEK1, Piccolo, liprin-α, and paxillin.

However, studies on GIT have remained insufficient thus far, particularly with no reports on relationship with GIT and cellular senescence.

### RELATED ART DOCUMENT

### Patent Literature

(Patent literature 1) Korean Patent No. 10-2019-0095502 A

### SUMMARY OF THE INVENTION

Herein, the present inventors first discovered the inhibitory activity of GIT against cellular senescence which has remained unveiled so far, on the basis of the finding that the down-regulation of βPIX (PAK-interacting exchange factor beta) promoted cellular senescence and reduced endocytosis and the senescent cells resulting from the down-regulation of βPIX decreased in the expression of senescence indices and were restrained from senescence-induced reduction of endocytosis, with the overexpression of GIT (G-protein-coupled receptor (GPCR) kinase-interacting proteins) therein, suggesting that GIT can be used as a cellular senescence inhibitor and ultimately as a novel therapeutic agent for senescence or senescence-associated diseases.

Therefore, a purpose of the present disclosure is to provide a pharmaceutical composition comprising GIT (G-protein-coupled receptor (GPCR) kinase-interacting proteins) or a GIT activating agent as an active ingredient for prevention or treatment of senescence or senescence-associated disease.

Another purpose of the present disclosure is to provide a composition comprising GIT (G-protein-coupled receptor (GPCR) kinase-interacting proteins) or a GIT activating agent as an active ingredient for inhibiting cellular senescence.

A further purpose of the present disclosure is to provide a method for screening a cellular senescence inhibitor.

A still further purpose of the present disclosure is to provide a method for inhibiting cellular senescence, the method comprising a step of treating cells with a GIT protein or an expressing vector carrying a GIT gene.

In order to achieve the above purpose thereof, the present disclosure provides a pharmaceutical composition comprising G-protein-coupled receptor (GPCR) kinase-interacting protein (GIT) or a GIT activating agent as an active ingredient for prevention or treatment of senescence or senescence-associated disease.

In an embodiment of the present disclosure, the GIT may comprise any one selected from the group consisting of the amino acid sequences of SEQ ID NOS: 1 to 4 and the GIT activating agent may be a protein, compound, or nucleic acid capable of enhancing GIT expression or activity. In an embodiment of the present disclosure, the GIT may be delivered in a form of a GIT gene inserted into an expression vector, the GIT gene being selected from the group consisting of the nucleotide sequences of SEQ ID NOS: 5 to 8.

In an embodiment of the present disclosure, the senescence or the senescence-associated disease may be caused by down-regulation of PAK1-interacting exchange factor beta (βPIX) expression.

In an embodiment of the present disclosure, the composition may have activity of inhibiting: SA-β-galactosidase activity; p16 expression; and cellular senescence-induced endocytosis reduction.

In addition, the present disclosure provides a composition comprising G-protein-coupled receptor (GPCR) kinase-interacting protein (GIT) or a GIT activating agent as an active ingredient for inhibiting cellular senescence.

In an embodiment of the present disclosure, the GIT may comprise any one selected from the group consisting of the amino acid sequences of SEQ ID NOS: 1 to 4 and the GIT activating agent may be a protein, compound, or nucleic acid capable of enhancing GIT expression or activity.

In an embodiment of the present disclosure, the GIT may be delivered in a form of a GIT gene inserted into an expression vector, the GIT gene being selected from the group consisting of the nucleotide sequences of SEQ ID NOS: 5 to 8.

In an embodiment of the present disclosure, the cellular senescence may be caused by down-regulation of PAK1-interacting exchange factor beta (βPIX) expression.

Furthermore, the present disclosure provides a method for screening a cellular senescence inhibitor, the method comprising the steps of: treating a biological sample with a candidate; detecting an expression level of a GIT protein or gene from the biological sample; and comparing the expression level with that of the same gene or protein in a control that has not been treated with the candidate.

In an embodiment of the present disclosure, the method may further comprise a step of determining the candidate as a cellular senescence inhibitor when the group treated with the candidate has an elevated expression level of the GIT protein or gene, compared to the control.

Moreover, the present disclosure provides a method for inhibiting cellular senescence, the method comprising a step of applying a GIT protein or an expression vector carrying a GIT gene to an isolated cell.

In an embodiment of the present disclosure, the GIT protein may comprise any one selected from the group consisting of the amino acid sequences of SEQ ID NOS: 1 to 4 and the GIT gene may comprise any one selected from the group consisting of the nucleotide sequences of SEQ ID NOS: 5 to 8.

### BRIEF DESCRIPTION OF THE DRAWINGS

The above and other aspects, features and advantages of the present disclosure will be more apparent from the following detailed description taken in conjunction with the accompanying drawings, in which:

FIG. 1 shows analysis results of expression levels of βPIX with age, illustrating (1A) expression levels of βPIX in tissues (lung, kidney, spleen, heart, and skin) from 3-, 15-, or 24-month-old mice as analyzed by immunoblotting, (1B) expression levels of βPIX and p16 in the lung tissue of each of the mice as analyzed by immunohistochemical staining, (1C) expression levels of βPIX and p16 as analyzed by immunohistohemical staining and depicted in a bar graph, (1D and 1E) expression levels of βPIX and p16 in lung tissues from young and old persons as measured by immunohistochemical staining and depicted in a bar graph, (1F) expression levels of βPIX and p16 in HDF cells different in the number of passages, as analyzed by immunohistochemical staining, (1G) results of SA-β-Gal activity assay, and (1H) activity levels of SA-3-Gal-positive cells in lung tissues from young and old persons as measured by immunohistochemical staining and depicted in a bar graph;

FIG. 2 shows reductive effects of the down-regulation of βPIX expression on endocytosis, illustrating (2A) images for integrin β1 uptake in HDF cells that are treated with siRNA for βPIX and then with nocodazole, as analyzed by staining with an anti-β1 integrin antibody, (2B) quantitation of endocytosed integrin β1, (2C) fluorescent images for transferrin endocytosis in HDF cells treated with siRNA for βPIX, (2D) quantitation of endocytosed transferrin, (2E) confocal microscopic images for transferrin endocytosis in lung tissues from mice injected with siRNA for βPIX, and (2F) quantitation of the transferrin endocytosis in a bar graph;

FIG. 3 shows association behaviors of protein complexes in focal adhesions upon cellular senescence induced by down-regulation of βPIX expression, illustrating (3A) the association of paxillin and calpain-2 in HDF cells treated with siRNA for βPIX, as analyzed by immunoprecipitation and immunoblotting, (3B) association behaviors of paxillin, calpain-2, and AMPH (amphiphysin) in βPIX- or GIT1/2-knockdown HDF cells, (3C) AMPH cleavage in GIT-knockdown HDF cells, (3D and 3E) quantitation of endocytosed transferrin (3D) and β1 integrin (3E) in GIT-knockdown HDF cells, (3F) association behaviors of GIT and paxillin in βPIX-knockdown HDF cells, (3G) localization of GIT and paxillin in HDF cells treated with Cy5-labeled siCtrl and siβPIX as analyzed by confocal microscopy using antibodies to paxillin and GIT, (3I) localization of calpain-2 and paxillin in cells, and (3H and 3J) quantitation of GIT and calpain-2/paxillin in focal adhesions (FA);

FIG. 4 shows association behaviors of AMPH-I and calpain-2 with the cell adhesion control protein paxillin, illustrating (4A) AMPH-I and calpain-2 being bound with paxillin as analyzed by immunoprecipitation and immunoblotting (anti-calpain 2: left, anti-AMPH-I: right), (4B) direct binding of paxillin to calpain-2 as analyzed by GST pulldown assay, (4C) direct binding of paxillin to AMPH-I as analyzed by GST pulldown assay, and (4D) a change in the association behavior of the protein complex in focal adhesions, depending on βPIX in a schematic view;

FIG. 5 shows association behaviors of GIT and calpain-2 with the cell adhesion control protein paxillin, illustrating (5A) competition of GIT1 and calpain-2 for binding to paxillin as analyzed by immunoblotting for binding of GIT and calpain-2 to paxillin after beads-bound paxillin was incubated with GST-calpain-2, and then various predetermined concentrations of GST-GIT-CT (C terminus) or GST were added, (5B) co-immunoprecipitaion of GFP-GIT-CT and paxillin after siβPIX-treated HDF cells were infected by lentivirus expressing GFP or GFP-GIT-CT, (5C) confocal images for localization of GIT-CT and paxillin in focal adhesions (FAs) of siβPIX-treated HDF cells, (5D) quantification of co-localized GFP-GIT-CT and paxillin, and (5E) quantification of cells positive for the cellular senescence marker SA-β-Gal after the cells were infected with lenti-GFP or lenti-GFP-GIT-CT virus prior to siβPIX treatment;

FIG. 6 shows inhibitory effects of GIT-CT (GIT-C terminus) overexpression on cellular senescence and endocytosis reduction, illustrating (6A) an experimental scheme conducted in mice, (6B and 6B) visualization and quantitation of transferrin uptake in lung tissues of siβPIX-treated mice after GFP or GFP-GIT-CT was expressed in the tissues, (6D) expression levels of the cellular senescence markers SA-β-Gal, p16 and βPIX, as analyzed by immunohistochemical staining, and (6E) expression quantitation of the markers;

FIG. 7 is a schematic view illustrating changes in the association behavior of protein complexes in focal adhesions (FA) with the expression of βPIX, accounting for the mechanism of the βPIX/GIT complex in cellular senescence, wherein an association behavior is given when the βPIX/GIT complex is sufficient (left), calpain-2 binds paxillin and cleaves AMPH when βPIX and/or GIT is depleted (middle), and the depletion of βPIX and/or GIT leads to senescence (right); and

FIG. 8 is a cleavage map of the recombinant expression vector pHR-CMV—SV-Puro-GIT1 C-terminus carrying the GIT1 C-terminus according to an embodiment of the present disclosure.

### DETAILED DESCRIPTION OF THE EXEMPLARY EMBODIMENTS

In the present disclosure, GIT (G-protein-coupled receptor (GPCR) kinase-interacting proteins) was first identified to have inhibitory activity against senescence.

Intensive studies conducted by the present inventors to develop a novel senescence inhibitor resulted in the finding that senescent cells have declined expression levels of βPIX (PAK1-interacting exchange factor beta) and that βPIX knockdown-induced cellular senescence is prevented by the overexpression of GIT.

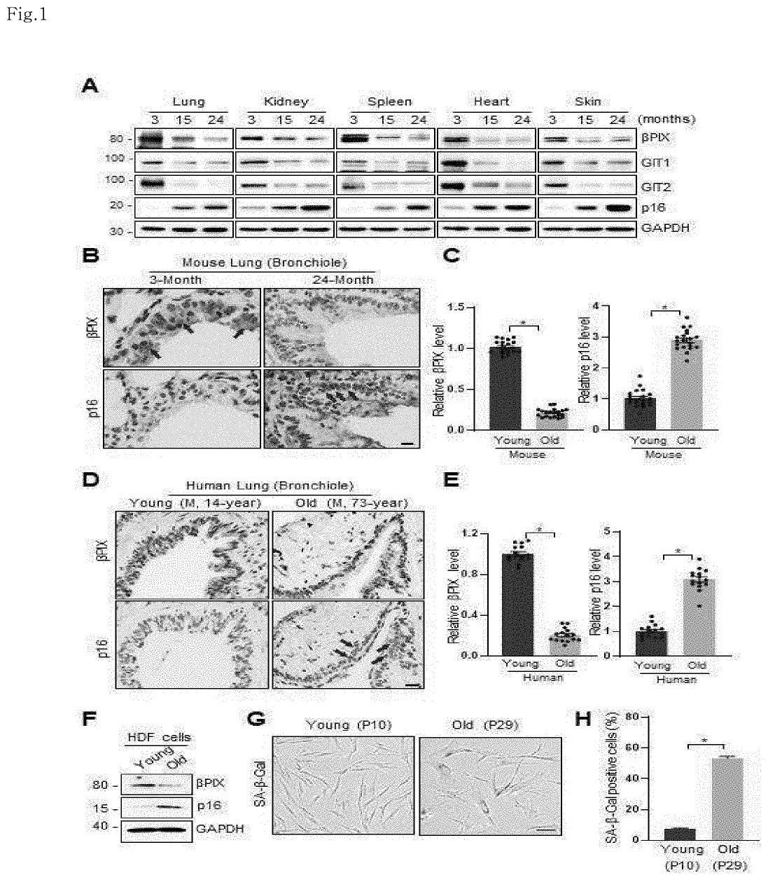

In this regard, according to an embodiment of the present disclosure, changes in the expression level of βPIX were assessed as a function of age. From data for expression levels of βPIX and GIT in the lung, kidney, spleen, heart, and skin tissues from 3-, 15-, or 24-month-old mice, it was discovered that expression levels of βPIX and GIT were remarkably decreased in all of the tissues from old mice, compared to young mice (see FIG. 1A), which was correspondingly coincident with the upregulated expression of the senescence marker p16.

Moreover, lung tissues from old mice and humans and old HDF cells were measured to have a markedly low expression levels of βPIX, but an increased expression level of the senescence marker p16 and an increases activity of SA-β-gal (senescence-associated β-galactosidase), compared to young lung tissues and cells (see FIG. 1B-1H). Therefore, it is understood that expression changes of βPIX and GIT are closely correlated with senescence, and reduced expression or activity of βPIX is mainly responsible for senescence.

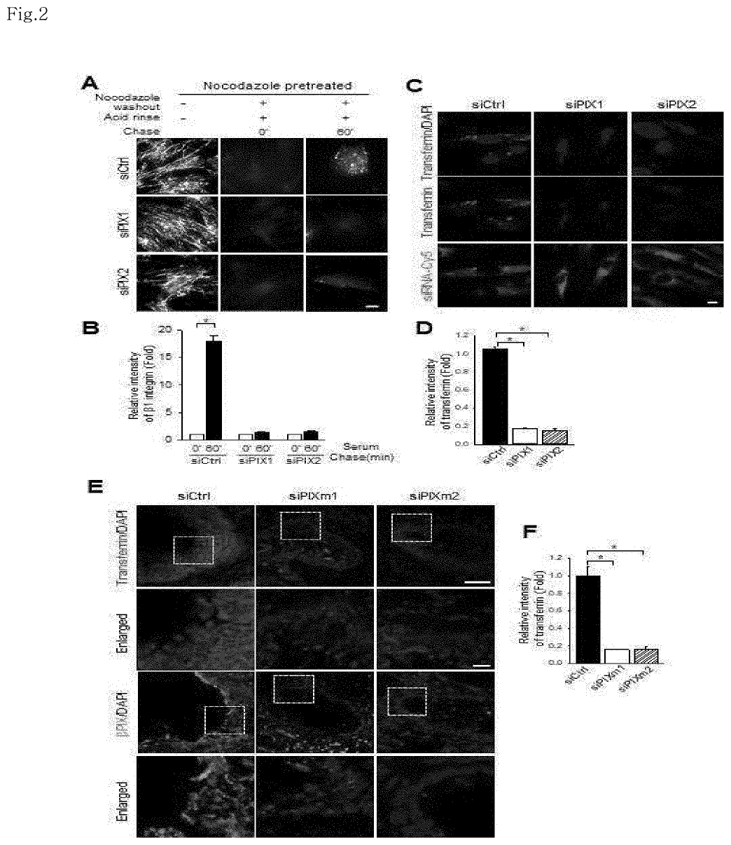

In another embodiment of the present disclosure, an analysis was made of relationship between cellular senescence and endocytosis. Senescent cells were less prone to integrin β1 and transferrin uptake (see FIG. 2). Thus, down-regulation of βPIX expression induces the upregulation of senescence markers and the reduction of endocytosis, leading to senescence.

Furthermore, cellular senescence is controlled by various mechanisms. Inter alia, the normal control of focal adhesion (FA) assembly and disassembly by regulatory proteins is involved in cellular senescence as well as growth and migration of cells. Hence, interaction between cell adhesion regulating proteins and cellular senescence proteins may be an important mechanism that can regulate senescence.

In the present disclosure, studies were conducted into interaction between GIT associating with the cell adhesion protein paxillin and bPIX acting as a member of the GIT complex, resulting in suggesting a novel use of GIT in regulating cellular senescence and identifying the mechanism of cellular senescence attributed to the down-regulation of bPIX expression.

An examination was made to see whether cellular senescence alters association behaviors of the protein complexes in FA. To this end, senescent cells induced by down-regulation of βPIX expression and young cells expressing βPIX were analyzed for association behaviors of paxillin, known as a focal adhesion adaptor protein, calpain-2, known as a protease, amphiphysin-I (AMPH-I), known as an endocytosis regulator, and GIT1/2.

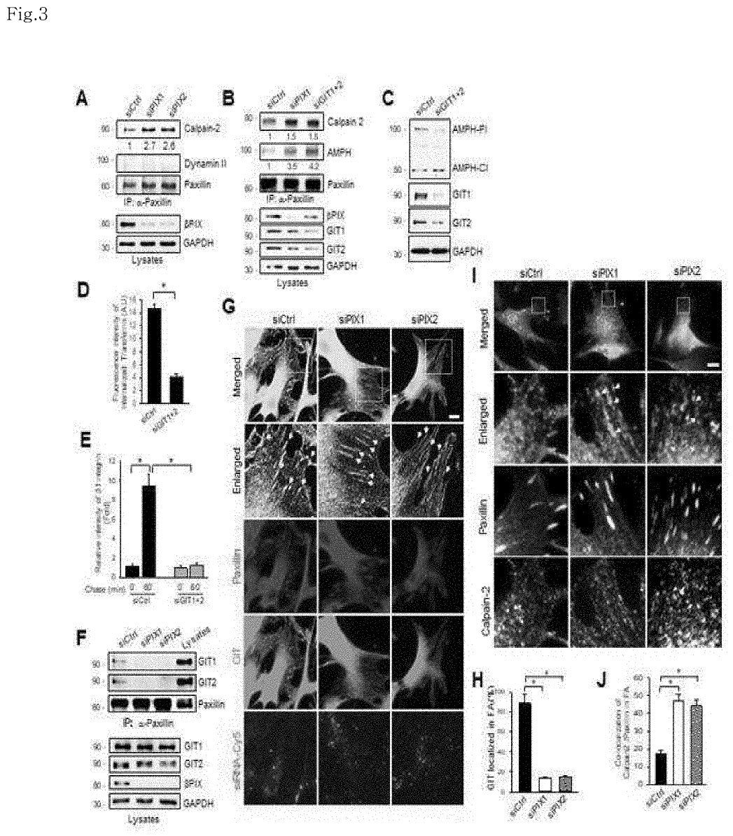

As a result, the down-regulation of bPIX expression promoted the association of calpain-2 and AMPH-I with paxillin. Promoted association of AMPH-1 with paxillin was also detected upon the down-regulation of GIT1/2 expression, with the concomitant cleavage of AMPH-I. In addition, GIT1/2-knockdown cells showed poor uptake of transferrin and β1 integrin (see FIG. 3A-3F). Furthermore, the down-regulation of βPIX expression inhibited the localization of GIT and calpain-2 to focal adhesions (FAs) (see FIG. 3G-3J).

It was observed that when cellular senescence was induced by the down-regulation of βPIX expression, an alteration was detected in the association behavior of proteins bound by the focal adhesion protein paxillin. In brief, the down-regulation of βPIX expression reduced the binding of paxillin to GIT1/2, but enhanced the binding of paxillin to calpain-2 and AMPH-I.

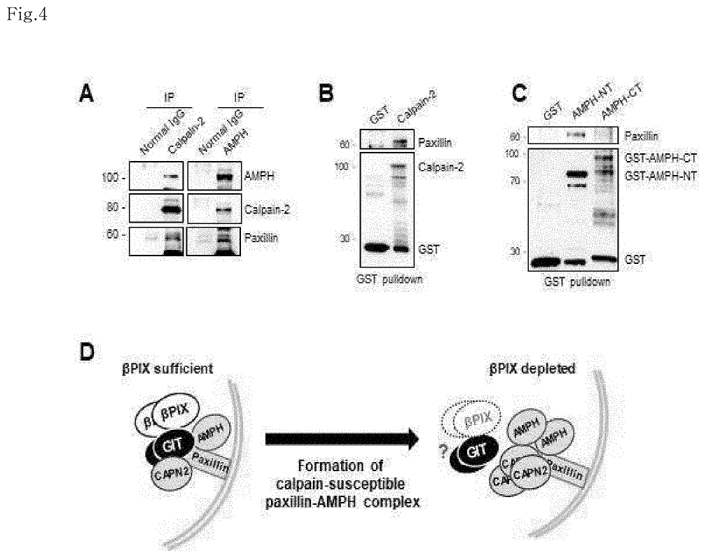

As the association of calpain-2 and AMPH-I with paxillin in focal adhesions was identified to increase with cellular senescence, the binding sites of paxillin, calpain-2, and AMPH-I were examined. In senescent cells having a down-regulated expression of βPIX, the three proteins paxillin, calpain-2, and AMPH-I were associated with one another, wherein paxillin was found to direct bind to the N terminus of AMPH-I (see FIG. 4). On the other hand, young cells expressing βPIX sufficiently was hypothesized to have a complex in which paxillin/GIT/βPIX/calpain-2/AMPH-I are suitably associated with one another (see FIG. 4D).

Furthermore, the present inventors made an examination to see whether or not GIT1 competes with calpain-2 for binding to paxillin. In this regard, the GIT C terminus that contains the paxillin-binding site, and calpain-2 were each overexpressed in bacteria to perform a competition assay.

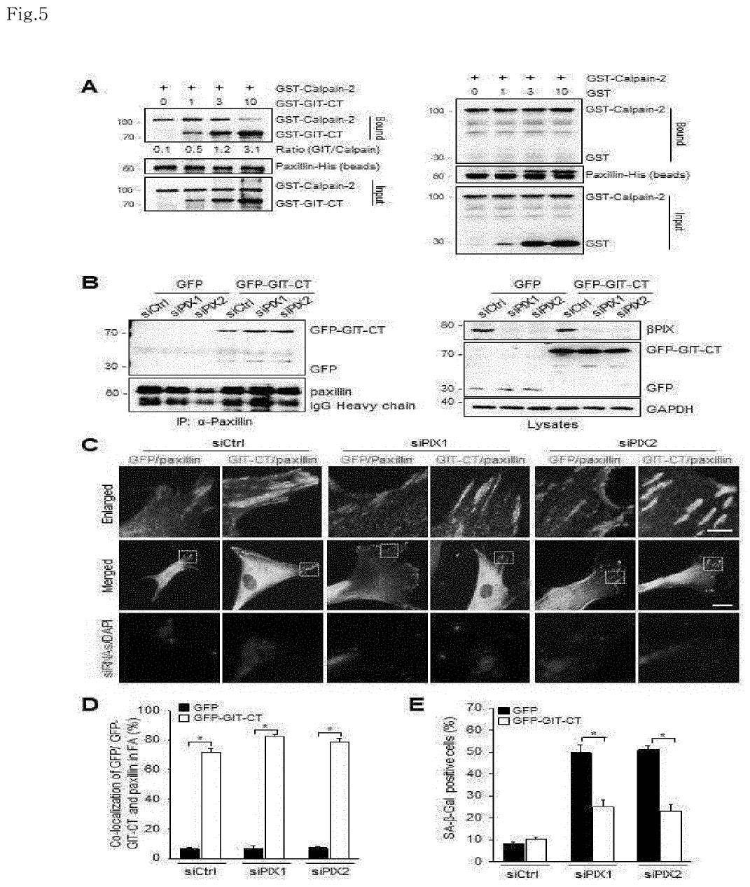

As a consequence, the more the GIT C terminus is added, the less the calpain-2 is bound by paxillin, indicating that GIT and calpain-2 complete with each other for association with paxillin (see FIGS. 5A and 5B).

In addition, GIT-CT (C-terminus) was observed to specifically co-localize with bPIX in focal adhesions even in the condition of down-regulated bPIX expression. Particularly, overexpression of GIT-CT can reverse the cellular senescence induced by the down-regulation of βPIX expression, as analyzed by SA-β-Gal staining (see FIGS. 5C-5E).

From the data thus obtained in the experiments in which the overexpression of GIT was first identified to inhibit the cellular senescence induced by the down-regulation of βPIX expression, the present inventors understood the use of GIT as a novel inhibitor against cellular senescence.

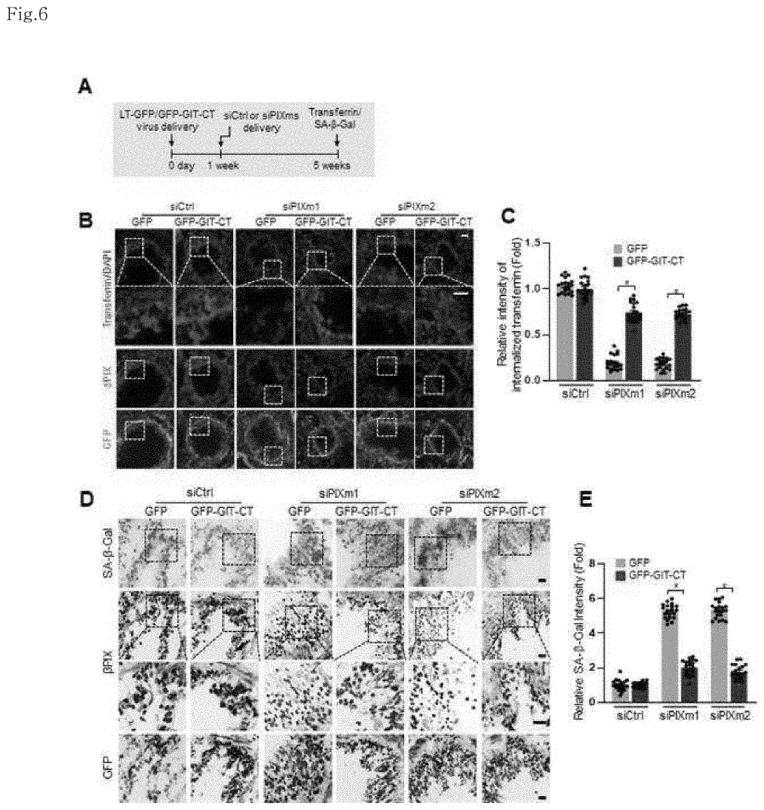

In addition, experiments with mouse lung tissues exhibited lower expression levels of SA-β-Gal and p16 in GIT-CT-overexpressed groups, compared to a control, with endocytosis reduction inhibited upon GIT-CT overexpression (see FIG. 6).

These data imply that GIT or a GIT activating agent can effectively inhibit the cellular senescence induced by the down-regulation of βPIX expression and as such, can be used as a novel therapeutic agent for senescence or senescence-associated disease.

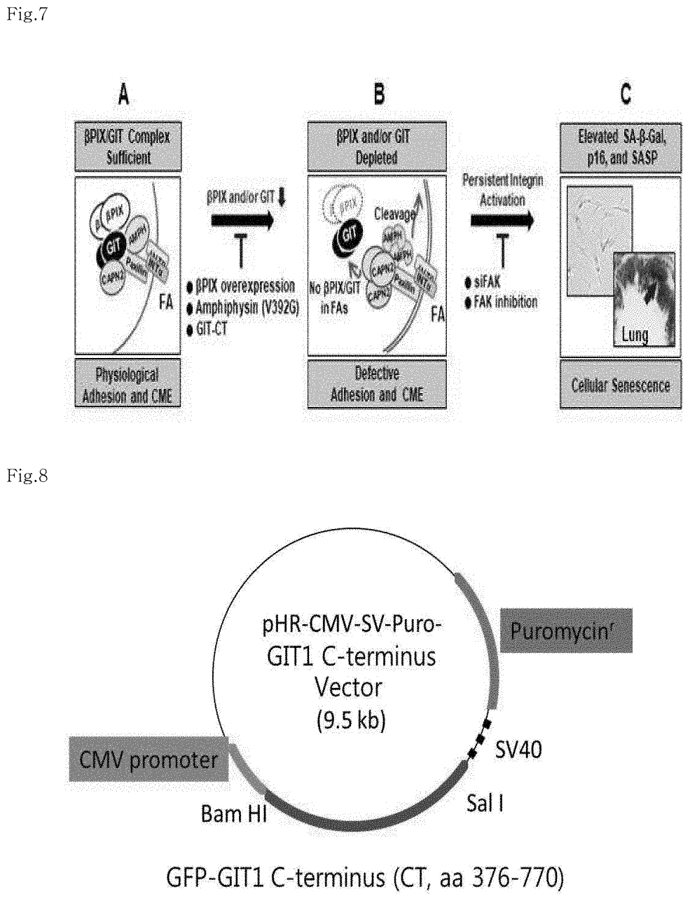

Led by considerations on the data, the present inventors analyzed association behaviors of the protein complexes in focal adhesions (FAs) as a function of βPIX expression (see FIG. 7). When βPIX is expressed sufficiently, the proteins in the focal adhesion are normally associated with paxillin to regulate cell adhesion and endocytosis (see FIG. 7A). On the other hand, when βPIX is depleted, GIT which forms a complex with βPIX leaves from paxillin (see FIG. 7B) and instead, the protease calpain-2 binds to paxillin and cleaves the C terminus of AMPH. Cleavage of AMPH C-terminus provokes a reduction in endocytosis and particularly induces the continuous activation of the integrin signaling system, leading to cellular senescence (see FIG. 7C).

Therefore, the present disclosure may provide a pharmaceutical composition comprising GIT (G-protein-coupled receptor (GPCR) kinase-interacting proteins) or a GIT activating agent as an active ingredient for prevention or treatment of senescence or senescence-associated disease.

In the present invention, the GIT(G-protein-coupled receptor (GPCR) kinase-interacting proteins) may comprise both of GIT1 and GIT2 and may be particularly composed of any one selected from the group consisting of the amino acid sequences of SEQ ID NOS: 1 to 4. Here, SEQ ID NO: 1 accounts for the full-length amino acid sequence of GIT1, SEQ ID NO: 2 for an amino acid sequence of GIT1 C-terminus, SEQ ID NO: 3 for the full-length amino acid sequence of GIT2, and SE ID NO: 4 for an amino acid sequence of GIT2 C-terminus. More particularly, the GIT may comprise the sequence of SEQ ID NO: 2 accounting for the GIT1 C terminus.

The GIT activating agent functions to promote the expression or activity of GIT and may be, but not limited to, a protein, a compound, or a nucleic acid specific for GIT.

In an embodiment of the present disclosure, the overexpression of GIT is achieved using an expression vector carrying a GIT gene composed of any one selected from the nucleotide sequences of SEQ ID NOS: 5 to 8.

Here, SEQ ID NO: 5 accounts for the full-length DNA sequence of GIT1, SEQ ID NO: 6 for a nucleotide sequence of the GIT1 C terminus, SEQ ID NO: 7 for the full-length DNA sequence of GIT2, and SEQ ID NO: 8 for a nucleotide sequence of the GIT2 C terminus.

Examples of the vector available in the present disclosure include, but are limited to, plasmids, phages, cosmids, viral vectors, and other mediators known in the art. In addition, the polynucleotide coding for GIT may be isolated from nature or may be artificially synthesized or modified. One or more bases of the nucleotide sequence coding for GIT may be modified by substitution, deletion, or addition as long as the modification does not result in a significant change in the biological activity of the protein expressed. Such modifications may include modifications into different homologous genes.

The expression vectors according to the present disclosure may be introduced into cells by using a method known in the art. For examples, vectors may be delivered into cells by transient transfection, microinjection, transduction, cell fusion, calcium phosphate precipitation, liposome-mediated transfection, DEAE Dextran-mediated transfection, polybrene-mediated transfection, electroporation, a gene gun, and other well-known intracellular delivery methods (Wu et al., J. Bio. Chem., 267:963-967, 1992; Wu and Wu, J. Bio. Chem., 263:14621-14624, 1988), but without limitations thereto.

Preferably, the GIT activating agent may be an expression vector having a GIT gene inserted thereto and particularly a lentiviral vector carrying a GIT gene.

The lentiviral vector of the present disclosure may be designed to have a GIT encoding gene operatively linked to a promoter.

As used herein, the term “operatively linked” refers to a functional linkage between a nucleic acid to be expressed and a nucleic acid expression regulatory sequence in such a manner as to allow the expression of the nucleic acid. As used herein, the term “promoter” refers to a DNA sequence capable of regulating the transcription of the nucleotide sequence of interest into mRNA, when ligated to a nucleotide sequence of interest. Typically, a promoter is, though not necessarily, located at the 5′ terminus (i.e., upstream) of a nucleotide sequence of interest whose transcription into mRNA is regulated thereby, and provides sites to which RNA polymerase and other transcription factors for initiation of transcription bind specifically.

The promoter of the present disclosure may be a constitutive promoter or a regulatable promoter, and particularly a constitutive promoter. The term “constitutive” when made in reference to a promoter means that the promoter is capable of directing transcription of an operably linked nucleic acid sequence in the absence of a stimulus (e.g., heat shock, chemicals, etc.). In contrast, a “regulatable” promoter is one which is capable of directing a level of transcription of an operably linked nucleic acid sequence in the presence of a stimulus (e.g., heat shock, chemicals, etc.) which is different from the level of transcription of the operably linked nucleic acid sequence in the absence of the stimulus.

In addition, the vector may further carry a gene coding for a fluorescent protein. The fluorescent protein aims to detect whether a gene of interest is expressed in a cell or tissue into the gene has been transduced. So long as it can fluoresce when expressed in cells or tissues, any fluorescent protein can be employed. Examples of the fluorescent protein include, but are not limited to, green fluorescent protein (GFP), modified green fluorescent protein, enhanced green fluorescent protein (EGFP), red fluorescent protein (RFP), enhanced red fluorescent protein (ERFP), blue fluorescent protein (BFP), enhanced blue fluorescent protein (EBFP), yellow fluorescent protein (YFP), enhanced yellow fluorescent protein (EYFP), cyan fluorescent protein (CFP), and enhanced cyan fluorescent protein (ECFP), with preference to green fluorescent protein (GFP).

The pharmaceutical composition of the present disclosure may contain a pharmaceutically acceptable carrier in addition to the active ingredient. A pharmaceutically acceptable carrier that is typically used in formulations may be available in the present disclosure. Examples of the pharmaceutically acceptable carrier include lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil, but are not limited thereto. The pharmaceutical composition of the present disclosure may further contain a lubricant, a wetting agent, a sweetening agent, a flavouring agent, an emulsifier, a suspension, a preservative, and the like, in addition to the above components. Appropriate pharmaceutically acceptable carriers and formulations are described in Remington's Pharmaceutical Sciences (19^{th} ed., 1995) in detail.

An appropriate dosage of the pharmaceutical composition of the present disclosure may be variously prescribed by factors such as formulation methods, administration types, age, body weight, sex, and morbidity of patients, food, administration time, administration route, excretion rate and response sensitivity. A daily dosage of the pharmaceutical composition of the present disclosure may be preferably 0.0001 to 100 mg/kg (body weight)

The pharmaceutical composition of the present invention may be orally or parenterally administered, and the parenteral administration may include intravenous injection, subcutaneous injection, intramuscular injection, intraperitoneal injection, transdermal administration, and the like. The concentration of an active ingredient in the composition of the present disclosure may be determined in view of therapeutic purposes, a patient's conditions, duration, or the like, but is not limited to a specific range.

The pharmaceutical composition according to the present disclosure may be formulated using a pharmaceutically acceptable carrier and/or an excipient according to a method easily executable by those skilled in the art and prepared in a unit dose form or be contained in a multi-dose container. In this case, the formulation may be a solution in oil or an aqueous medium, a suspension or emulsion, an extract, a powder, granules, a tablet, or a capsule, and may further include a dispersing or stabilizing agent.

As used herein, the term “treatment”, unless stated otherwise, refers to the action that can reverse or relieve the disease or disorder itself or one or more symptoms of the disease or disorder targeted by the term, or that can inhibit the progression of the disease or disorder or prevent the onset of the disease or disorder.

The disease in the present disclosure may be senescence or a senescence-associated disease. For example, the disease may be the senescence which is caused with the inhibition of βPIX (PAK1-interacting exchange factor beta) expression or may be a senescence-associated disease which enters an onset stage with the progression of senescence.

Examples of the senescence-associated disease include, but are not limited to, atherosclerosis, skin aging, osteoporosis, rheumatoid osteoarthritis, degenerative osteoarthritis, alopecia, wrinkles, and a humpback.

In addition, the composition of the present disclosure can prevent or treat senescence or a senescence-associated disease through the activity of suppressing SA-β-galactosidase activity; down regulating p16 expression; and inhibiting cellular senescence-induced endocytosis reduction.

Moreover, the present disclosure provides a composition comprising GIT (G-protein-coupled receptor (GPCR) kinase-interacting proteins) or a GIT activating agent as an active ingredient for inhibiting cellular senescence.

As described in the foregoing, the upregulation of GIT expression in cells or tissues was identified to lead to a reduction of senescence.

In addition, the present disclosure may provide a method for screening a cellular senescence inhibitor, the method comprising the steps of: treating a biological sample with a candidate; detecting an expression level of a GIT protein or gene from the biological sample; and comparing the expression level with that of the same gene or protein in a control that has not been treated with the candidate.

The method may further comprise a step of determining the candidate as a cellular senescence inhibitor when the group treated with the candidate has an elevated expression level of the GIT protein or gene, compared to the control.

Here, the biological sample may be a tissue or cell.

The detection or measurement of an expression level of GIT protein may be carried out by Western blotting (immunoblotting), enzyme linked immunosorbent assay (ELISA), radioimmunoassay (RIA), radioimmunodiffusion, ouchterlony immunodiffusion, rocket immunoelectrophoresis, immunohistochemical staining, immunoprecipitation assay, complement fixation assay, fluorescence activated cell sorter (FACS), or protein chip, without limitations thereto.

An expression level of GIT gene can be measured using reverse transcription polymerase chain reaction (PCR), competitive PCR, real-time PCR, nuclease protection assay (RNase, S1 nuclease assay), in situ hybridization, DNA microarray, or northern blotting, without limitations thereto.

Moreover, the present disclosure provides a method for inhibiting cellular senescence, the method comprising a step of applying a GIT protein or an expressing vector carrying a GIT gene to isolated cells.

That is, a GIT protein or an expression vector carrying a GIT gene is delivered into tissues or cells to increase GIT expression, thereby inhibiting cellular senescence.

A better understanding of the present disclosure may be obtained through the following examples which are set forth to illustrate, but are not to be construed as limiting the present disclosure.

### PREPARATION EXAMPLES

Materials and Methods

Materials and experiment methods used in the experiments and assays of the following Examples were as follows.

Materials

Invivofectamine, Lipofectamine 2000, Dulbecco's modified Eagle's medium (DMEM), fetal bovine serum (FBS), Opti-MEM, Alexa Fluor 594-conjugated transferrin, and Alexa Fluor-conjugated secondary antibodies were purchased from Thermo Fisher Scientific (Waltham, Mass.).

shRNA lentiviruses for p16 and p53 were purchased from Santa Cruz Biotechnology (Dallas, Tex.). Lentiviruses for amphiphysins, si-rPIXs, and GIT-CT were provided by Dr. Sung from Korea Research Institute of Bioscience and Biotechnology (Cheongju, Korea). Lentiviruses for GFP (LVP690) and GFP-βPIX (LVP718951) were purchased from Abm Inc. (Richmond, Canada).

Calpain inhibitors II (ALLM), SA-β-Gal staining solution, and other chemicals were purchased from Sigma-Aldrich (St. Louis, Mo.).

Recombinant paxillin-His protein was purchased from RayBiotech (Peachtree Corners, Ga.).

Human inflammation antibody array was purchased from Abcam (Cambridge, UK).

siRNAs for GIT1(SI02224467) and GIT2 (HSS114794) were obtained from Qiagen (Germantown, Md.). siRNAs for βPIX, calpain-2, and siFAK were obtained from Thermo Fisher Scientific and Bioneer (Daejeon, Korea).

siRNA or shRNA sequences employed in the assays are summarized as follows:

siPIX1:
(SEQ ID NO: 9)
5′-UCAACUGGUAGUAAGAGCAAAGUUU-3′
siPIX2:
(SEQ ID NO: 10)
5′-UUGAGCUGCAGAUCCUGACGGAAGC-3′
siCtrl:
(SEQ ID NO: 11)
5′-CCUACGCCACCAAUUUCGU-3′
hCalpain-2:
(SEQ ID NO: 12)
5′-GGCAUUAGAAGAAGCAGGUUU-3′
siPIXr1:
(SEQ ID NO: 13)
5-ACUGGUAGUACGAGCCAAGUU-3′
siPIXr2:
(SEQ ID NO: 14)
5-GGAGGAUUAUGAUCCUGAUAG-3′
siPIXm1:
(SEQ ID NO: 15)
51-CCAACUGGUAGUACGAGCCAAGUUU-3′
siPIXm2:
(SEQ ID NO: 16)
5′-GAGGACCUAGGAGAGUUCAUGGAAA-3′
siFAK:
(SEQ ID NO: 17)
5′-AACCACCUGGGCCAGUAUUAU-3′

Animals

Animal experiments were performed in accordance with the approved animal protocols and the guidelines established by the Ethics Review Committee of the Chungbuk National University for Animal Experiments (CBNUA-901-15-01). Mice were obtained from DahanBioLink (Seoul, Korea).

Tissue Samples

Human lung cells and tissues were obtained from patients with pneumothorax who underwent surgical operation at the Chungbuk National University Hospital (Cheongju, Korea). These patients did not show any other pathology in the lungs. All of the studies were reviewed and approved by the Institutional Review Board of the Chungbuk National University Hospital (2014-02-009-009).

Antibodies

Anti-pFAK (Y576) (#3281; 1:500), FAK (#3258; 1:1000 for immunoblotting/1:200 for immunohistochemistry), p53 (#2524; 1:1000), pp53 (S15) (#9284; 1:500), pPAK1 (T423) (#2610; 1:500), paxillin (Y118) (#2541; 1:500), and pyH2AX (S139) (#9713; 1:200) antibodies were purchased from Cell Signaling Technology (Danvers, Mass.). Anti-pFAK (Y397) (611806; 1:500), paxillin (610051; 1:1000), GIT2 (P94020; 1:1000), Cdk2 (610145; 1:500), Cdk4 (610147; 1:500), Cyclin D (610279; 1:500), Cyclin E (551159; 1:500), pRB (610884; 1:500), ppRB (610490; 1:500), and p19 (610530; 1:500) antibodies were purchased from BD Biosciences (San Jose, Calif.). Antibodies against calpain-2 (sc-373966; 1:1000) and calpain-4 (sc-30065; 1:1000), p16 (sc-28260; 1:500 for immunoblotting/1:200 for immunohistochemistry), p21 (sc-6246; 1:500), GIT1 (sc-9657; 1:500), and amphiphysin I (sc-376402 and sc-39028; 1:1000) were purchased from Santa Cruz Biotechnology (Dallas, Tex.).

GFP (NB600-308; 1:1000 for immunoblotting/1:200 for immunohistochemistry) antibody was purchased from Novus Biologicals (Centennial, Colo.). The active integrin β₁ (MAB2079Z; 1:50) and GST (A00895; 1:1000) antibodies were purchased from Merck Millipore (Burlington, Mass.) and GenScript (Piscataway, N.J.), respectively. Anti-β₁ integrin antibody (ab30394; 1:50), and 6×His-tag (ab18184; 1:1000) antibodies were from Abcam (Cambridge, UK). Anti-βPIX antibody (1:1000 for immunoblotting/1:200 for immunohistochemistry) was raised against the C-terminal region (a.a. 439-648) of βPIX.

Plasmids and DNA Constructs

βPIX constructs, si-rPIX (WT), and si-rPIX (DHmt) were cloned into pHR-CMV SV40 for lentiviral expression. Mutagenesis for si-rPIX (WT) and si-rPIX constructs was performed using a QuikChange II site-directed mutagenesis kit (Agilent). N-terminal (NT, a.a. 1-351) and C-terminal (CT, a.a. 346-695) amphiphysin I constructs and calpain-2 were cloned into pGEX4T-1. Wild-type (WT) and mutant (MT (V392G)) constructs of amphiphysin I were cloned into pHR-CMV SV40 for lentiviral expression. Calpain-2 was cloned into pGEX4T-1. Calpain-2 complementary DNA was purchased from OriGene. GIT1 C-terminus (CT, a.a. 376-770) was cloned into pGEX4T-1 for bacterial expression and pHR-CMV SV40 for lentiviral expression, respectively. A cleavage map of the recombinant expression vector pHR-CMV—SV-Puro-GIT1 C-terminus carrying the GIT1 C-terminus according to an embodiment of the present disclosure is depicted in FIG. 8.

In Vivo Delivery of siRNA, Lentivirus, or Transferrin

Mice were anesthetized with avertin (2, 2, 2-tribromoethanol, 0.45 mg/g of body weight) by intraperitoneal injection and placed on a platform that held the mouse's top front teeth on the bar. A 2.54-cm, 22-gauge Safelet IV catheter with blunted needle was located into the trachea by peering into the mouth and looking for white light emission from the trachea. After the catheter was ensured to be in the trachea, the blunted needle was removed from the catheter. The Invivofectamine-RNA interference complex (75 μl of liposomes) was prepared according to the manufacturer's protocol, and the in vivo delivery of lentivirus particles or Alexa Fluor 594-conjugated transferrin was achieved by directly pipetting into the opening of the catheter.

Cell Culture

HDF (human diploid fibroblast) and 293T cells were cultured in DMEM (Dulbecco's modified Eagle's medium) supplemented with 10% FBS and antibiotics in a 5% CO₂ incubator at 37° C.

SA-β-Gal Assay

SA-β-Gal activity was measured at pH 6.0 as described in Proc Natl Acad Sci USA 92, 9363-9367 (1995), with slight modifications. Briefly, cells were washed with phosphate-buffered saline (PBS), fixed with 3% formaldehyde for 5 min, and washed with PBS. Cells were then incubated in SA-β-Gal staining solution (Sigma-Aldrich) for 13-14 hours at 37° C. and stained with Hoechst 33258 for 30 min before cell counting. Cellular senescence was scored as a percentage of SA-β-Gal-positive cells (blue staining) relative to the total count of cells. For tissues, animals were anesthetized and perfused with saline. Tissues were flash-frozen in liquid nitrogen and embedded into an OCT compound. The tissues were immediately cut into 10-μm sections, fixed with 1% formaldehyde in PBS, washed with PBS, and incubated in SA-β-Gal staining solution for 13-14 hours at 37° C. Thereafter, the nuclei were stained with Safranin-O and mounted with Vectashield mounting medium (Vector Laboratories Inc., Burlingame, Calif.).

In Vitro Binding Assay

GST-tagged or 6×His-tagged proteins were purified with glutathione or Ni-NTA affinity chromatography, respectively. Purified proteins were incubated on binding buffer (20 mM HEPES, pH 7.4, 0.15 M NaCl, 1 mM DTT, 0.2% Triton X-100, 5 μM MgSO₄, and protease inhibitor) for 30 min at room temperature. Beads were washed five times with binding buffer and then subjected to SDS-PAGE before immunoblotting with a specific antibody.

Focal Adhesion Analysis

HDF cells were plated on fibronectin-coated coverslips. After 1 day, the cells were transfected with siRNAs and then treated with control or RGD peptides for 3 days. The cells were stained with antibody for paxillin and Alexa Fluor 568-conjugated phalloidin, followed by observing FAs under Olympus FluoView FV1000, or 1λ81-ZDC inverted microscope (Olympus, Japan) equipped with a cool charge-coupled device camera, Cascade 512B (Photometrics). FA number and actin intensity were determined using ImageJ software.

Transient Transfection

Transfection of DNA or siRNA was performed using Lipofectamine 2000 or Lipofectamine RNAiMAX transfection reagent according to the manufacturer's instruction. Cells were seeded in plates or glass coverslips coated with fibronectin. Transfection into cells was carried out with DNA for 1 day or with siRNA for 3-4 days.

Immunohistochemistry

Tissues were fixed with 10% neutral buffered formalin, dehydrated, and embedded in paraffin. Sections (4 μm thick) were cut from formalin-fixed, paraffin-embedded tissue blocks. After deparaffinization, slides were subjected to an antigen retrieval procedure in 10 mM sodium citrate buffer (pH 6.0) for 10 min using a pressure cooker (Dedoaking Chamber, Biocare Medical), followed by incubation with a blocking solution (0.3% Triton X-100, 1% bovine serum albumin, 0.05% Tween 20, 0.1% cold-water fish gelatin, and 0.05% sodium azide in PBS) for 1 hour at room temperature. Primary antibodies were incubated overnight with the sections at 4° C. Following five washes with 0.1% Tween 20, 0.1% BSA in PBS, each slide was incubated with an Alexa Fluor-conjugated secondary antibody (1:200) for 1 hour in a dark chamber at room temperature. The slides were washed many times, counterstained with Hoechst 33258, and analyzed with Vectashield mounting medium (Vector Laboratories Inc.). For DAB (diaminobenzidine-HCl) staining, slides were incubated in methanol containing 0.3% hydrogen peroxide for 20 min at room temperature to block endogenous peroxidase activity before applying a blocking solution. Then, the slides were incubated with a biotin-conjugated secondary antibody for 30 min at room temperature and lastly with peroxidase-conjugated streptavidin for 30 min at room temperature. Peroxidase activity was measured using the substrate DAB. For negative controls, sections were treated with TBS without a primary antibody. For histologic evaluation, sections were stained with H&E.

Immunocytochemistry

Cells were fixed for 15 min with 3.7% paraformaldehyde, permeabilized for 5 min with 0.2% Triton X-100, and blocked for 30 min at 25° C. with 2% BSA in PBS. For antigen staining, the cells were incubated with a primary antibody for 1 hour at 25° C., followed by incubation with a secondary Alexa Fluor-conjugated antibody for 1 hour. To visualize F-actin, the cells were stained with Alexa Fluor 568-conjugated phalloidin for 30 min at 25° C. Expression of stained proteins was analyzed by MetaMorph software version 7.1.7 (Molecular Devices).

Immunoblotting and Immunoprecipitation

Cells were lysed with cold lysis buffer (50 mM HEPES, pH 7.5, 150 mM NaCl, 10% glycerol, 1% Triton X-100, 500 μM EDTA, 200 μM sodium pyruvate, and 50 mM β-glycero-phosphate), and the supernatants were immunoprecipitated with the indicated primary antibody for 18 hours at 4° C. The immunoprecipitates were separated by 8 to 10% SDS-PAGE and transferred to a polyvinylidene fluoride membrane in a tris-glycine-methanol buffer (25 mM tris base, 200 mM glycine, and 20% methanol). The membrane was blocked with 3% BSA in TBS (TBS-T; 50 mM tris, 150 mM NaCl, and 0.1% Tween 20) for 30 min, incubated with a primary antibody for 1 hour at room temperature, and washed three times with TBS-T. Then, the membrane was incubated with a secondary horseradish peroxidase-conjugated antibody for 1 hour at room temperature and washed three times with TBS-T. Signals were detected using an enhanced chemiluminescence reagent.

Transferrin Endocytosis

Cells were incubated on ice for 10 min in cold live cell image solution (LCIS; 140 mM NaCl, 20 mM HEPES, 2.5 mM KCl, 1.8 mM CaCl₂), and 1.0 mM MgCl₂, pH 7.4) containing 20 mM glucose and 1% BSA. The cells were incubated with Alexa Fluor 594-transferrin (20 μg/ml) in LCIS containing 20 mM glucose and 1% BSA for 15 min at 37° C., washed with PBS, and then fixed with 4% paraformaldehyde for 10 min. To analyze transferrin endocytosis in mouse lung, 0.1 μM Alexa Fluor 594-transferrin was inserted directly into the opening of the catheter for intratracheal delivery. After 1 hour, the animals were perfused with PBS through the heart. The tissues were dissected, flash-frozen in liquid nitrogen, and then immediately sliced into a thickness of 10 μm with the cryomicrotome before fixing with cold acetone for 10 min. The sections were stained with DAPI (10 μg/ml) to label the nucleus and mounted with Vectashield mounting medium. Endocytotic transferrin was observed with Olympus FluoView confocal microscope (FV10i, Olympus, Japan) or fluorescence microscope (DP30BW, Olympus, Japan), and fluorescence intensity was analyzed with ImageJ software.

Integrin β1 Endocytosis

Starved cells were pretreated with 10 μM nocodazole for 20 min and incubated with anti-active β₁ integrin antibody (1:200) for 40 min at 37° C. Unbound antibodies and nocodazole were washed away with PBS, and a 60-min chase was performed. Then, the cells were washed with warm PBS, followed by acid rinse (0.5% acetic acid and 0.5 M NaCl, pH 3.0) to remove surface antibodies. The cells were fixed with 4% paraformaldehyde and permeabilized with 0.2% Tween 20. Internalized active β₁ integrin was analyzed by incubation with Alexa Fluor-conjugated secondary antibody (1:200) for 1 hour.

GST-PBD Pulldown Assay

Glutathione-Sepharose bound GST-PBD proteins were incubated in siRNA-treated cell lysates for 20 min at room temperature and washed five times with PBS. GST-PBD Sepharose beads were resolved by 12% SDS-PAGE, transferred to PVDF membranes, and immunoblotted with antibodies

Statistical Analysis

Data are expressed as means±SEM; representative data from at least three independent experiments were analyzed. Statistical significance was assessed by unpaired Student's t test, Wilcoxon-Mann-Whitney test, or One Way ANOVA using SigmaPlot (version 12) for Windows. P<0.05 was considered statistically significant.

### Example 1

Analysis of βPIX Expression Level in Senescent Tissue and Cell

In order to assess expression levels of βPIX as a function of age, lung, kidney, spleen, heart, and skin tissues from 3-, 15-, or 24-month-old mice were analyzed for protein expression levels of bPIX and GIT by immunoblotting. As a result, the tissues (lung, kidney, spleen, heart, and skin) of old mice were measured to have significantly reduced protein expression levels of bPIX and GIT and an elevated level of the senescence marker p16, compared to young mice (see FIG. 1A).

For more detailed examination, expression levels of bPIX and p16 protein, a senescence marker, in lung tissues from young mice (3 months old) and old mice (24 months old) were analyzed by immunohistochemical staining. The expression of bPIX was remarkably reduced in lungs from the old mice, compared to the young mice whereas the expression of the senescence marker p16 was elevated in the old mice (see FIGS. 1B and 1C). These results were coincident with those obtained by immunohistochemical staining for lung tissues from young persons (10's) and old persons (70's). The lung tissues from old persons exhibited reduced expression levels of bPIX, but elevated expression levels of p16 protein (see FIGS. 1D and 1E).

In addition, examination was made to see whether the same results can be obtained at cell levels. In this regard, βPIX and p16 expression levels were measured in young passage human dermal fibroblast (HDF) cells and old passage human dermal fibroblast (HDF) cells. A reduced expression level of bPIX was detected in old HDF cells, compared to young HDF cells whereas an elevated expression level of p16 was measured in the old HDF cells. SA-β-gal (senescence associated-β-galactosidase), also known as a senescence marker, was measured to exhibit elevated activity in old HDF cells (see FIGS. 1F-1H). Thus, expression GIT as well as bPIX was understood to decline with age, leading to the conclusion that reduced expression levels of βPIX-GIT are closely associated with senescence.

### Example 2

Reduction of Integrin β1 and Transferrin Endocytosis by Down-Regulation of βPIX

As examined in Example 1, senescence provokes a reduction in βPIX expression. Through the following experiments, investigation was made to see whether endocytosis works as a senescence mechanism responsible for the down-regulated expression of βPIX.

In order to investigate how the down-regulation of βPIX influences the endocytosis of integrin β1, which regulates cell adhesion, human dermal fibroblasts (HDF) were treated with siRNA for βPIX and nocodazole and then incubated with an anti-active integrin β1 antibody. Internalized integrin β1 was analyzed by a staining method using the anti-active integrin β1 antibody.

Furthermore, to analyze how the down-regulation of βPIX expression influences transferrin endocytosis, human dermal fibroblasts (HDF) were treated with siRNA for βPIX, followed by quantitating internalized Alexa Fluor 594-transferrin to analyze endocytotic transferrin. In addition, lung tissues of mice were treated with siRNA for βPIX before analyzing the endocytosis of Alexa Fluor 594-Transferrin in bronchioles in the mouse lungs.

The siRNAs used for down-regulation of PM expression are as follows:

siPIX1:
(SEQ ID NO: 9)
5′-UCAACUGGUAGUAAGAGCAAAGUUU-3′
siPIX2:
(SEQ ID NO: 10)
5′-UUGAGCUGCAGAUCCUGACGGAAGC-3′
siCtrl:
(SEQ ID NO: 11)
5′-CCUACGCCACCAAUUUCGU-3′
siPIXm1:
(SEQ ID NO: 15)
5′-CCAACUGGUAGUACGAGCCAAGUUU-3′
siPIXm2:
(SEQ ID NO: 16)
5′-GAGGACCUAGGAGAGUUCAUGGAAA-3′

As an analysis result, the endocytosis of the cell adhesion controlling protein integrin β 1 in HDF cells was remarkably decreased where bPIX expression was down regulated, compared to HDF cells in which bPIX expression was not down regulated (siCtrl treated group) (see FIGS. 2A and 2B). Transferrin, which is a representative marker for endocytosis, was endocytosed at remarkably decreased rates in bPIX-downregulated groups (see FIGS. 2C and 2D). This result was consistent with that in the mouse lung tissues injected with siRNA for βPIX. βPIX-knockdown tissues were less prone to transferrin endocytosis (see FIGS. 2E and 2F). Accordingly, these data indicate that the down-regulation of βPIX expression may promote senescence which results in a decrease in endocytosis.

### Example 3

Interaction Between GIT and Focal Adhesion Protein Upon Down-Regulation of βPIX Expression

Cells that had undergone senescence by treatment with siRNA for βPIX were observed for changes in proteins in focal adhesions (FAs). To this end, immunoprecipitation was carried out with an antibody to paxillin, a representative FA protein, to analyze changes of paxillin complexes in senescent cells.

In senescence-induced cells by down-regulation of βPIX expression, it was observed that the proteinase calpain-2 and the endocytosis regulator AMPH-I (amphiphysin-I) highly associated with paxillin (see FIGS. 3A and 3B). In addition, when the expression of GIT1/2, which acts in association with βPIX, was inhibited by treatment with siRNA for GIT1/2, the association of AMPH with paxillin was increased and AMPH-I was cleaved (see FIG. 3C).

Furthermore, the down-regulation of GIT1/2 by treatment with siRNA specific for GIT1/2 decreased endocytosis of transferrin and integrin (see FIGS. 3D and 3E). In the cellular senescence condition of PM down-regulation, a remarkably reduced level of GIT1/2, which is bound with the cell adhesion protein paxillin, was detected (see FIG. 3F).

In addition, localization of the cell adhesion protein paxillin and GIT1/2 in bPIX-downregulated senescent cells was analyzed. In this regard, HDF cells were treated with Cy5-labeled siCtrl (control) or siβPIX, and stained with antibodies to paxillin, GIT, and calpain-2, followed by localization of each protein in focal adhesion (FA) through a confocal microscope.

As a result, βPIX-downregulated senescent cells showed increased co-localization of the paxillin-GIT complex, but decreased co-localization of the paxillin-calpain-2 complex in focal adhesions (FAs) (see FIGS. 3G-3J).

It was understood from the result that cellular senescence altered localization of the protein complexes and the proteins in FAs. Particularly in βPIX-downregulated senescent cells, association was observed to decrease between the cell adhesion protein paxillin and GIT1/2, but increase between calpain-2 and AMPH.

### Example 4

Analysis of Association Behavior of AMPH-I (Amphiphysin-I) and Calpain-2 with Cell Adhesion Protein Paxillin

To analyze association behaviors of AMPH-I and calpain-2 with the cell adhesion protein paxillin, HDF cells were subjected to immunoprecipitation with antibodies to calpain-2 and AMPH-I, followed by immunoblotting for paxillin. As a result, the three proteins paxillin, AMPH-I, and calpain-2 were detected to exist as complexes therebetween (see FIG. 4A).

In greater detail, association between paxillin and calpain-2 was analyzed by GST pulldown assay after the proteins were overexpressed in bacteria. This assay confirmed association between paxillin and calpain-2 was observed (see FIG. 4B). In addition, a binding site of AMPH-1 to paxillin was examined. Following overexpression of AMPH-N-terminus (NT) and AMPH-C-terminus (CT) regions, a GST pulldown assay revealed that AMPH-1 directly binds to paxillin through the AMPH-N terminus (see FIG. 4C).

From the data, it was understood that association behaviors of proteins with paxillin in FA are dependent on the expression of βPIX. Based on this result, the schemes of FIG. 4D for association behaviors of proteins in cells could be derived.

That is, pre-senescent cells in which βPIX is sufficiently expressed allow paxillin/GIT/βIX/calpain/AMPH to be suitably associated with each other to form complexes. When the expression of βPIX decreases with age, GIT leaves, together with βPIX, from paxillin in FA and instead, more calpain-2 and AMPH-I proteins rush to the same sites to form paxillin/calpain/AMPH complexes. In this context, calpain-2 is predicted to cleave AMPH-I to inhibit the clathrin-dependent endocytosis of integrin β1 and transferrin, thus inducing cellular senescence. In addition, the data indicate that when βPIX is expressed, GIT competes with calpain-2 for a binding site to paxillin (see FIG. 4D).

### Example 5

Analysis of Association Behavior of GIT and Calpain-2 with Cell Adhesion Protein Paxillin

In order to observe whether association of GIT and calpain-2 with the cell adhesion control protein paxillin is regulated depending on the expression level of βPIX, GIT and calpain-2 were analyzed for competition for binding to paxillin. The GIT1-C-terminus (GIT-CT, amino acids 376-770), known as a binding site for paxillin, and calpain-2 were overexpressed in bacteria. Then, Ni²⁺ bead-bound paxillin was incubated with GST-calpain-2. In this regard, the GST-GIT1-CT protein was added in an amount of 0, 1, 3, and 10 μg, GST-calpain-2 associated with paxillin was quantitated by immunoblotting. The amount of paxillin-bound calpain-2 was decreased with an increase in the amount of GST-GIT-CT added while no different association was observed upon addition of elevated amounts of GST protein as a control (see FIG. 5A).

Furthermore, under a bPIX knockdown condition by treatment with siRNA bPIX, HDF cells were induced to overexpress GFP and GFP-GIT-CT and subjected to co-immunoprecipitation with an antibody to paxillin, followed by immunoblotting. This assay revealed the direct binding of paxillin to GIT-CT (see FIG. 5B) and the specific co-localization of GIT-CT and paxillin in FA (see FIGS. 5C and 5D). In addition, when induced to overexpress GIT-CT, the βPIX-knockdown HDF cells were observed to reduce the activity of SA-β-gal (see FIG. 5F).

These results imply that GIT overexpression can effectively inhibit the cellular senescence induced by down-regulation of bPIX expression.

### Example 6

Inhibitory Effect of GIT Overexpression on Cellular Senescence and Reduced Endocytosis in Animal Model

The ability of GIT overexpression to inhibit cellular senescence was identified through the in vitro cell experiment of Example 5. Then, the inhibitory activity of GIT overexpression against cellular senescence and reduced endocytosis was assayed in vivo in mice. For this, lentivirus vectors carrying a GFP-GIT-CT gene and a GFP gene (control) were constructed, respectively and then injected into mouse lungs. After one week, siβPIX and siCtrl were delivered to the mice. Four weeks after siRNA delivery, the mouse lung tissues were analyzed for cellular senescence and endocytosis. The experimental schedule is summarized in the schematic diagram of FIG. 6A.

The reduced transferrin endocytosis induced by the down-regulation of βPIX in the mouse lung tissues was remarkably inhibited with the overexpression of GFP-GIT-CT (see FIGS. 6B and 6C). GFP-GIT-CT-overexpressed groups were also measured to remarkably decrease in expression levels of the cellular senescence markers SA-β-Gal and p16, compared to the control (see FIGS. 7D and 7E).

### Example 7

Controlling Mechanism for βPIX Down-Regulation-Induced Senescence

From the data obtained above, therefore, it was understood that in the presence of sufficient βPIX, that is, in the pre-senescent condition, FA proteins remain normally associated with paxillin to control focal adhesions and endocytosis. In contrast, when βPIX expression is reduced by senescence, GIT complexed with βPIX leaves from focal adhesions while the protease calpain-2 increasingly binds thereto, instead, with the resultant cleavage of AMPH-I. The cleavage of AMPH-I incurs reduced endocytosis and particularly induces continuous activation of the integrin signaling system while promoting cellular senescence (see FIG. 7).

Consequently, the experimental results obtained in the present disclosure are the first evidence for identifying a concrete mechanism for cell adhesion-endocytosis-cellular senescence, which has remained unknown thus far. Especially, the full-length GIT protein or the GIT-CT protein can be used as a cellular senescence inhibitor as βPIX knockdown-induced cellular senescence has been identified to be inhibited by GIT overexpression.

As described hitherto, the present disclosure, which is the first report on the inhibitory activity of GIT against cellular senescence, reveals that senescent cells induced by down-regulation of bPIX expression are induced to reduce in the expression of senescence markers and cease senescence-induced endocytosis reduction by GIT overexpression therein, identifying correlation cell adhesion and endocytosis with βPIX knockdown-induced cellular senescence. Having an inhibitory activity against cellular senescence, GIT is suggested to be used as a novel therapeutic agent for senescence or senescence-associated disease according to the present disclosure.

What is claimed is:

1: A method of reducing cellular senescence in a mammal in need thereof, the method comprising administering to the mammal a composition comprising G-protein-coupled receptor (GPCR) kinase-interacting protein (GIT) or a GIT activating agent as an active ingredient.

2: The method of claim 1, wherein the GIT comprises any one selected from the group consisting of the amino acid sequences of SEQ ID NOS: 1 to 4 and the GIT activating agent is a protein, compound, or nucleic acid capable of enhancing GIT expression or activity.

3: The method of claim 1, wherein the GIT is delivered in a form of a GIT gene inserted into an expression vector, the GIT gene being selected from the group consisting of the nucleotide sequences of SEQ ID NOS: 5 to 8.

4-5. (canceled)

6: A method of treating a disease or a disease symptom associated with increased cellular senescence in a mammal with said disease or disease symptom, the method comprising administering to a mammal a composition comprising G-protein-coupled receptor (GPCR) kinase-interacting protein (GIT) or a GIT activating agent as an active ingredient.

7: The method of claim 6, wherein the GIT comprises any one selected from the group consisting of the amino acid sequences of SEQ ID NOS: 1 to 4 and the GIT activating agent is a protein, compound, or nucleic acid capable of enhancing GIT expression or activity.

8: The method of claim 6, wherein the GIT is delivered in a form of a GIT gene inserted into an expression vector, the GIT gene being selected from the group consisting of the nucleotide sequences of SEQ ID NOS: 5 to 8.

9. (canceled)

10: A method of reducing a SA-β-galactosidase activity and p16 expression in a mammalian cell, the method comprising administering to a mammal in need thereof a composition comprising G-protein-coupled receptor (GPCR) kinase-interacting protein (GIT) or a GIT activating agent as an active ingredient.

11: The method of claim 10, wherein the GIT comprises any one selected from the group consisting of the amino acid sequences of SEQ ID NOS: 1 to 4 and the GIT activating agent is a protein, compound, or nucleic acid capable of enhancing GIT expression or activity.

12: The method of claim 10, wherein the GIT is delivered in a form of a GIT gene inserted into an expression vector, the GIT gene being selected from the group consisting of the nucleotide sequences of SEQ ID NOS: 5 to 8.

13. (canceled)


SEQUENCE LISTING (17 sequences)

SEQ ID NO: 1 - protein, 770 aa, Artificial Sequence
  GIT1 full length amino acid sequence
MSRKGPRAEVCADCSAPDPGWASISRGVLVCDECCSVHRSLGRHISIVKHLRHSAWPPTLLQMVHTLASNGANSIWEHSLLDPAQVQSGRRKANPQDKVHPIKSEFIRAKYQMLAFVHKLPCRDDDGVTAKDLSKQLHSSVRTGNLETCLRLLSLGAQANFFHPEKGTTPLHVAAKAGQTLQAELLVVYGADPGSPDVNGRTPIDYARQAGHHELAERLVECQYELTDRLAFYLCGRKPDHKNGHYIIPQMADRSRQKCMSQSLDLSELAKAAKKKLQALSNRLFEELAMDVYDEVDRRENDAVWLATQNHSTLVTERSAVPFLPVNPEYSATRNQGRQKLARFNAREFATLIIDILSEAKRRQQGKSLSSPTDNLELSARNQSDLDDQHDYDSVASDEDTDQEPLPSAGATRNNRARSMDSSDLSDGAVTLQEYLELKKALATSEAKVQQLMKVNSSLSDELRKLQREIHKLQAENLQLRQPPGPVPVPSLPSERAEHTLMGPGGSTHRRDRQAFSMYEPGSALKPFGGAPGDELATRLQPFHSTELEDDAIYSVHVPAGLYRIRKGVSASSVTFTPSSPLLSSSQEGSRHASKLSRHGSGAESDYENTQSGEPLLGLEGKRFLELSKEDELHAELESLDGDPDPGLPSTEDVILKTEQVTKNIQELLRAAQEFKHDSFVPCSEKIHLAVTEMASLFPKRPALEPVRSSLRLLNASAYRLQSECRKTVPPEPGAPVDFQLLTQQVIQCAYDIAKAAKQLVTITTREKKQ

SEQ ID NO: 2 - protein, 395 aa, Artificial Sequence
  GIT1 C terminal amino acid sequence
LELSARNQSDLDDQHDYDSVASDEDTDQEPLPSAGATRNNRARSMDSSDLSDGAVTLQEYLELKKALATSEAKVQQLMKVNSSLSDELRKLQREIHKLQAENLQLRQPPGPVPVPSLPSERAEHTLMGPGGSTHRRDRQAFSMYEPGSALKPFGGAPGDELATRLQPFHSTELEDDAIYSVHVPAGLYRIRKGVSASSVTFTPSSPLLSSSQEGSRHASKLSRHGSGAESDYENTQSGEPLLGLEGKRFLELSKEDELHAELESLDGDPDPGLPSTEDVILKTEQVTKNIQELLRAAQEFKHDSFVPCSEKIHLAVTEMASLFPKRPALEPVRSSLRLLNASAYRLQSECRKTVPPEPGAPVDFQLLTQQVIQCAYDIAKAAKQLVTITTREKKQ

SEQ ID NO: 3 - protein, 759 aa, Artificial Sequence
  GIT2 full length amino acid sequence
MSKRLRSNDVCADCSGPDPSWASVNRGTLICDECCSVHRSLGRHISQVRHLKHTPWPPTLLQMVETLYSNGANSIWEHSLLDPASVMSGRRKANPQDKVHPNKAEFIRAKYQMLAFVHRLPCRDDDSVTAKDLSKQLHSSVRTGNLETCLRLLSLGAQANFFHPEKGSTPLHVASKAGQILQAELLAVYGADPGTHDSSGKTPVDYARQGGHRELAERLVEIQYELTDRLAFYLCGRKPDHKNGQHFIIPQMADSSLDLSELAKAAKKKLQSLSNHLFEELAMDVYDEVDRRETDAVWLATQNHSTLVTETTVVPFLPVNPEYSSTRNQGRQKLARFNAHEFATLVIDILSDAKRRQQGSPLSRSKDNVELILRTVSNQHSTESQDNDQPDYDSVASDEDTDVETRASRTNRQKSLDSDLSDGPVTVQEFMEVKHALVASEAKRQQLMKVNNNLSGELRIMQKKLQTLQSENSSLRRQATASACQVQTASDHKDTVSHSSLKRRPSARGSRPMSMYETGSGQKPYLPMGEANHPEESRTRLQPFPTHIGRSALVTSSSSLPSFPSTLSWSRDESTRRASRLEKQNSTPESDYDNTAYDPEPDDTVSGRKGRQRSMLWQGDGPLPDTAEPHAVPSPALPSTEDVIRKTEQITKNIQELLRAAQENKHDSYIPCSERIHAAVTEMAALFPKKPKSDTVRTSLRLLTASAYRLQSECRKALPGDSSLPTDVQLVTQQVIQCAYDIAKAAKQLVTITTKENSS

SEQ ID NO: 4 - protein, 384 aa, Artificial Sequence
  GIT2 C terminal amino acid sequence
VSNQHSTESQDNDQPDYDSVASDEDTDVETRASRTNRQKSLDSDLSDGPVTVQEFMEVKHALVASEAKRQQLMKVNNNLSGELRIMQKKLQTLQSENSSLRRQATASACQVQTASDHKDTVSHSSLKRRPSARGSRPMSMYETGSGQKPYLPMGEANHPEESRTRLQPFPTHIGRSALVTSSSSLPSFPSTLSWSRDESTRRASRLEKQNSTPESDYDNTAYDPEPDDTVSGRKGRQRSMLWQGDGPLPDTAEPHAVPSPALPSTEDVIRKTEQITKNIQELLRAAQENKHDSYIPCSERIHAAVTEMAALFPKKPKSDTVRTSLRLLTASAYRLQSECRKALPGDSSLPTDVQLVTQQVIQCAYDIAKAAKQLVTITTKENSS

SEQ ID NO: 5 - DNA, 2313 bp, Artificial Sequence
  GIT1 full length DNA sequence
atgtcccggaaggggccgcgagcggaggtgtgtgcggactgcagcgcccctgaccctggctgggcatctatcagcagaggtgtgctggtttgtgacgagtgctgcagtgtgcaccggagcctgggacgacacatctccattgtcaaacaccttcgccacagcgcctggcctcctacgctgctacagatggtgcacacgcttgccagcaacggggccaactctatctgggagcattccctgctggaccctgcacaagtgcaaagtggccggcgcaaagccaacccccaagacaaagtccaccccatcaagtcagagttcatcagggcaaagtaccagatgctggcgtttgtgcacaagcttccctgccgtgatgatgatggggtcaccgccaaagacctcagcaagcaactgcactcgagtgtgcgaacaggcaacctggagacctgtctgcgcctgctttccctgggtgcccaggccaacttcttccacccagaaaagggcactacacctctacacgtggctgccaaggcagggcagacactgcaggctgagctgctagtagtgtatggggctgacccaggctcccctgatgtcaatggccgcacacccattgactatgccaggcaggcggggcaccatgaactggcagaaaggctagttgagtgccagtatgagctcactgaccggttggccttctacctctgtggacggaagcctgatcacaagaatgggcattacatcataccacagatggctgacagatctcggcaaaagtgcatgtctcagagtttggatctgtctgaattggccaaagctgccaagaagaagctacaagcactcagcaaccggctctttgaggagctcgccatggatgtgtatgacgaagtggaccggagagaaaatgatgcagtgtggctagctacccagaatcatagcaccctggtgacagaacgcagtgctgtgcccttcctgccagtcaaccctgaatactcagccactcggaatcagggacgacagaaattggctcgttttaatgcccgagagtttgccaccttgatcatcgacatcctcagtgaggccaaacggaggcagcagggcaagagtctgagcagccccacagacaacctcgagctgtctgcacggaaccaaagtgacctggacgaccagcacgactacgatagtgtggcttctgacgaagacacagaccaggagcccttgcccagtgctggcgccacgcggaacaatcgtgccaggagcatggactcctcagatctgtcggatggggctgtgacgctgcaggagtacctggagctgaagaaggctctggctacctccgaggccaaagtgcagcagctcatgaaagtcaacagcagtctgagtgacgagcttcggaagctgcagagggagatccacaaactgcaggcagagaacctgcagctccggcagccaccaggaccagtgcctgtaccctcacttcccagtgaacgggcagaacacacactcatgggccctggtggaagtacccatcgcagggaccgccaggccttctctatgtatgagccaggctccgccctgaagccctttggaggtgcacctggggatgagctcgccacacggctccagcctttccacagcacagagctggaagatgatgccatctattcagtacatgtccctgctggcctttaccggatccggaagggggtgtctgcctcttctgtgaccttcactccctcctccccactgctgtcaagctcccaagaaggaagtcgccatgcgagcaagctttcacgccacggcagtggcgcagagagtgactatgagaacacacagagtggagagcctctgctgggacttgaagggaagcgattcctagagctgagcaaggaggatgagctgcacgctgagctggagagcttagatggagacccagaccccgggctccccagcacagaagatgtcatcctaaagacagagcaggtcaccaagaacattcaggagctattgcgggcagcccaggagttcaaacatgacagctttgtgccctgttcagaaaagatccatttggctgtgactgagatggcctctctcttcccaaagaggccagccctggagcctgtgcgcagttcactgcggctgctcaacgccagcgcctaccggctgcagagtgaatgccggaagacagtgcctccagaacctggcgcccctgtggacttccagctactgactcagcaggtgatccagtgcgcctatgacatcgccaaggctgccaagcagctggtcaccatcaccacccgagagaagaaacagtga

SEQ ID NO: 6 - DNA, 1188 bp, Artificial Sequence
  GIT1 C terminal DNA sequence
ctcgagctgtctgcacggaaccaaagtgacctggacgaccagcacgactacgatagtgtggcttctgacgaagacacagaccaggagcccttgcccagtgctggcgccacgcggaacaatcgtgccaggagcatggactcctcagatctgtcggatggggctgtgacgctgcaggagtacctggagctgaagaaggctctggctacctccgaggccaaagtgcagcagctcatgaaagtcaacagcagtctgagtgacgagcttcggaagctgcagagggagatccacaaactgcaggcagagaacctgcagctccggcagccaccaggaccagtgcctgtaccctcacttcccagtgaacgggcagaacacacactcatgggccctggtggaagtacccatcgcagggaccgccaggccttctctatgtatgagccaggctccgccctgaagccctttggaggtgcacctggggatgagctcgccacacggctccagcctttccacagcacagagctggaagatgatgccatctattcagtacatgtccctgctggcctttaccggatccggaagggggtgtctgcctcttctgtgaccttcactccctcctccccactgctgtcaagctcccaagaaggaagtcgccatgcgagcaagctttcacgccacggcagtggcgcagagagtgactatgagaacacacagagtggagagcctctgctgggacttgaagggaagcgattcctagagctgagcaaggaggatgagctgcacgctgagctggagagcttagatggagacccagaccccgggctccccagcacagaagatgtcatcctaaagacagagcaggtcaccaagaacattcaggagctattgcgggcagcccaggagttcaaacatgacagctttgtgccctgttcagaaaagatccatttggctgtgactgagatggcctctctcttcccaaagaggccagccctggagcctgtgcgcagttcactgcggctgctcaacgccagcgcctaccggctgcagagtgaatgccggaagacagtgcctccagaacctggcgcccctgtggacttccagctactgactcagcaggtgatccagtgcgcctatgacatcgccaaggctgccaagcagctggtcaccatcaccacccgagagaagaaacagtga

SEQ ID NO: 7 - DNA, 2280 bp, Artificial Sequence
  GIT2 full length DNA sequence
atgtcgaagcgtctccggagcaacgacgtgtgcgccgactgcagcgggcctgatccttcctgggcatcagtgaacagggggactctcatatgtgatgagtgctgcagtgttcatcggagcctagggcgccacatctcccaagtcaggcatcttaaacacacaccatggcctccaaccctgcttcagatggttgagaccttgtatagcaatggtgctaattctatctgggagcattctttgctggaccctgcttctgttatgagtgggagacggaaagccaatccacaggataaagtccaccccaataaagcagaattcatcagagccaagtatcagatgttagcatttgtacacagattgccctgccgtgacgacgacagtgtgactgccaaagatcttagtaagcaactccattcaagtgtgagaacagggaatcttgagacctgtctgagactgttgtcgttaggagcccaagccaacttcttccatcctgaaaaaggaagcactccactccacgtcgcctccaaagcaggacagatcctacaggctgaattgctggcagtgtacggagctgacccaggcacacacgattctagtgggaagactcctgttgactatgcaaggcaaggaggacaccgtgagctggccgaacgcctcgtagaaatacagtacgagctgacggacaggctggccttctacctctgcggccggaaaccagatcataaaaacggacagcactttataatccctcagatggcagacagcagcctggatttgtctgagttggcaaaagctgccaagaagaaacttcagtctctaagtaatcatttgtttgaagaacttgccatggatgtgtacgatgaagttgacagacgagagactgatgcagtctggcttgccacgcaaaaccacagcaccttggtaactgagacaaccgtcgttccctttcttccggtcaaccctgagtactcctcgacacgaaaccagggcagacagaaattagctcggtttaatgcccatgaatttgctacactggtcatcgacatcctcagtgacgccaagaggagacagcaaggcagtcctctctccaggtcaaaagacaatgtggaactcatattgagaacagtcagtaaccagcacagcactgagagccaggacaatgaccagccagactatgacagcgtggcctcagatgaagacacagacgtggagaccagagcaagcaggacaaaccggcagaagagcctagattcagatttgtcagatggaccagtcactgtgcaggagtttatggaggtcaaacacgccctagtggcttctgaggccaagagacagcagctaatgaaggtgaataacaacctgagtggcgagctgagaattatgcagaaaaagcttcaaactctacagagtgagaactcaagcctcaggagacaggccacagccagtgcctgtcaggtccagactgcctctgaccacaaagacactgtgagccactcctccctgaagcgccggccatcagcccggggcagtagacccatgtccatgtatgagactggatcaggccagaaaccatacctcccaatgggagaagccaaccaccctgaagaaagccggacaagacttcagcccttccccactcatattgggaggagtgcacttgtgacctcctcttcgtctctgccttccttcccctccacactttcctggtccagggacgaaagcacccgaagggcctccaggttggagaagcagaacagcaccccagagagcgattatgacaacactgcctatgaccctgaaccagatgacacggtgtcaggccggaaggggcggcagcggagtatgctctggcaaggtgatggcccactgccagacacagcagagccccacgcggtccccagccccgccctccccagcacagaagatgtcatccgcaagactgaacagatcaccaaaaacatccaggagctcctcagagcagcccaggagaacaagcatgacagctacattccctgctcagagaggatacacgccgctgtgaccgagatggcagccctgttccccaaaaaacctaagtctgacacggtgaggacttccctccgcctactaacggccagtgcctaccggctccagtcagagtgcagaaaggctctcccaggagactcgagcttgcccacggatgtgcagctggtcacacagcaggtcatccagtgtgcctatgacattgccaaggccgccaaacagctggtcacaatcaccaccaaagagaacagcagctga

SEQ ID NO: 8 - DNA, 1155 bp, Artificial Sequence
  GIT2 C terminal DNA sequence
gtcagtaaccagcacagcactgagagccaggacaatgaccagccagactatgacagcgtggcctcagatgaagacacagacgtggagaccagagcaagcaggacaaaccggcagaagagcctagattcagatttgtcagatggaccagtcactgtgcaggagtttatggaggtcaaacacgccctagtggcttctgaggccaagagacagcagctaatgaaggtgaataacaacctgagtggcgagctgagaattatgcagaaaaagcttcaaactctacagagtgagaactcaagcctcaggagacaggccacagccagtgcctgtcaggtccagactgcctctgaccacaaagacactgtgagccactcctccctgaagcgccggccatcagcccggggcagtagacccatgtccatgtatgagactggatcaggccagaaaccatacctcccaatgggagaagccaaccaccctgaagaaagccggacaagacttcagcccttccccactcatattgggaggagtgcacttgtgacctcctcttcgtctctgccttccttcccctccacactttcctggtccagggacgaaagcacccgaagggcctccaggttggagaagcagaacagcaccccagagagcgattatgacaacactgcctatgaccctgaaccagatgacacggtgtcaggccggaaggggcggcagcggagtatgctctggcaaggtgatggcccactgccagacacagcagagccccacgcggtccccagccccgccctccccagcacagaagatgtcatccgcaagactgaacagatcaccaaaaacatccaggagctcctcagagcagcccaggagaacaagcatgacagctacattccctgctcagagaggatacacgccgctgtgaccgagatggcagccctgttccccaaaaaacctaagtctgacacggtgaggacttccctccgcctactaacggccagtgcctaccggctccagtcagagtgcagaaaggctctcccaggagactcgagcttgcccacggatgtgcagctggtcacacagcaggtcatccagtgtgcctatgacattgccaaggccgccaaacagctggtcacaatcaccaccaaagagaacagcagctga

SEQ ID NO: 9 - RNA, 25 nt, Artificial Sequence
  siPIX1
ucaacugguaguaagagcaaaguuu

SEQ ID NO: 10 - RNA, 25 nt, Artificial Sequence
  siPIX2
uugagcugcagauccugacggaagc

SEQ ID NO: 11 - RNA, 19 nt, Artificial Sequence
  siCtrl
ccuacgccaccaauuucgu

SEQ ID NO: 12 - RNA, 21 nt, Artificial Sequence
  hCalpain-2
ggcauuagaagaagcagguuu

SEQ ID NO: 13 - RNA, 21 nt, Artificial Sequence
  siPIXr1
acugguaguacgagccaaguu

SEQ ID NO: 14 - RNA, 21 nt, Artificial Sequence
  siPIXr2
ggaggauuaugauccugauag

SEQ ID NO: 15 - RNA, 25 nt, Artificial Sequence
  siPIXm1
ccaacugguaguacgagccaaguuu

SEQ ID NO: 16 - RNA, 25 nt, Artificial Sequence
  siPIXm2
gaggaccuaggagaguucauggaaa

SEQ ID NO: 17 - RNA, 21 nt, Artificial Sequence
  siFAK
aaccaccugggccaguauuau